(19) United States
(12) Patent Application Publication
(10) Pub. No.: US 2025/0166799 A1
(43) Pub. Date: May 22, 2025

(54) IMAGE CALIBRATION METHOD, IMAGE PROCESSING METHOD, ELECTRONIC DEVICE, AND STORAGE MEDIUM

(71) Applicant: EVision Technology (Beijing) Co., Ltd., Beijing (CN)

(72) Inventors: Saiguang LING, Beijing (CN); Zhou DONG, Beijing (CN); Xin KE, Beijing (CN); Jiqun DONG, Beijing (CN)

(73) Assignee: EVision Technology (Beijing) Co., Ltd., Beijing (CN)

(21) Appl. No.: 19/031,450

(22) Filed: Jan. 18, 2025

Related U.S. Application Data
(63) Continuation of application No. PCT/CN2023/126884, filed on Oct. 26, 2023.

(30) Foreign Application Priority Data
Nov. 2, 2022 (CN) 202211363571.2

(51) Int. Cl. G16H 30/40; G06T 7/11; G06T 7/62; G06V 10/25; G06V 10/56

(52) U.S. Cl. CPC G16H 30/40; G06T 7/11; G06T 7/62; G06V 10/25; G06V 10/56; G06T 2207/30041; G06V 2201/03

(57) ABSTRACT

Disclosed are an image calibration method and apparatus, an image processing method and apparatus, an electronic device, and a storage medium. The image calibration method includes: determining, based on a target fundus image, a papilla region of the target fundus image; determining, based on the target fundus image, an effective imaging region of the target fundus image, where the effective imaging region includes a region where a fundus structure is visualized; and determining, based on the effective imaging region and the papilla region, a calibration result of the target fundus image. By calibrating the target fundus image through the effective imaging region and the papilla region, comparison between fundus images captured by different cameras may be achieved, which helps to measure and study related fundus characteristics.

### CROSS-REFERENCE TO RELATED APPLICATIONS

This application is a continuation of International Application No. PCT/CN2023/126884, filed on Oct. 26, 2023, which claims priority to Chinese Patent Application No. 202211363571.2, filed on Nov. 2, 2022. All of the aforementioned applications are incorporated herein by reference in their entireties.

### TECHNICAL FIELD

The present disclosure relates to the field of image calibration technologies, and in particular, to an image calibration method, an image processing method, an electronic device, and a storage medium.

### BACKGROUND

A fundus image may be used to understand morphology of a fundus structure and changes in fundus characteristics, so that the fundus image has become an essential auxiliary tool for clinical disease diagnosis and treatment. With the advent of the big data era, researches on eye diseases and therapeutic effects have been increasingly conducted through a large volume of fundus images.

However, in production processes of fundus cameras, different manufacturers set different imaging parameters for their cameras. As a result, sizes of fundus characteristics captured by cameras with different imaging parameters may vary. This inconsistency makes it difficult to compare fundus images captured by cameras with different imaging parameters, which poses challenges for related researches.

### SUMMARY

In view of this, embodiments of the present disclosure provide an image calibration method and apparatus, an image processing method and apparatus, an electronic device, and a storage medium to solve a problem of difficulty in comparing different fundus images captured by different fundus cameras.

In a first aspect, an embodiment of the present disclosure provides an image calibration method, including: determining, based on a target fundus image, a papilla region of the target fundus image; determining, based on the target fundus image, an effective imaging region of the target fundus image, where the effective imaging region includes a region where a fundus structure is visualized; and determining, based on the effective imaging region and the papilla region, a calibration result of the target fundus image, where the calibration result includes a calibration result of a pixel unit dimension of the target fundus image and a calibration result of a fundus characteristic dimension of the target fundus image.

In a second aspect, an embodiment of the present disclosure provides an image processing method. The image processing method includes: calibrating a plurality of fundus images to be calibrated by the image calibration method according to the first aspect to generate calibration results respectively corresponding to the plurality of fundus images to be calibrated; determining, based on the calibration results respectively corresponding to the plurality of fundus images to be calibrated, a plurality of dimensional calibration results for a same fundus characteristic; and comparing the plurality of dimensional calibration results for the same fundus characteristic to obtain a comparison result of the plurality of dimensional calibration results.

In a third aspect, an embodiment of the present disclosure provides an image calibration apparatus, including: a first determination module, configured to determine, based on a target fundus image, a papilla region of the target fundus image; a second determination module, configured to determine, based on the target fundus image, an effective imaging region of the target fundus image, where the effective imaging region includes a region where a fundus structure is visualized; and a calibration module, configured to determine, based on the effective imaging region and the papilla region, a calibration result of the target fundus image, where the calibration result includes a calibration result of a pixel unit dimension of the target fundus image and a calibration result of a fundus characteristic dimension of the target fundus image.

In a fourth aspect, an embodiment of the present disclosure provides an image processing apparatus, including: a calibration module, configured to calibrate a plurality of fundus image data to be calibrated by the image calibration method according to the first aspect to generate calibration results respectively corresponding to the plurality of fundus image data to be calibrated; a determination module, configured to determine, based on the calibration results respectively corresponding to the plurality of fundus image data to be calibrated, a plurality of dimensional calibration results for a same fundus characteristic; and a comparison module, configured to compare the plurality of dimensional calibration results for the same fundus characteristic to obtain a comparison result of the plurality of dimensional calibration results.

In a fifth aspect, an embodiment of the present disclosure provides an electronic device, including: a processor; and a memory, configured to store executable instructions of the processor, where the processor is configured to implement the image calibration method according to the first aspect.

In a sixth aspect, an embodiment of the present disclosure provides a computer-readable storage medium, on which computer executable instructions are stored, where when the executable instructions are executed by a processor, the image calibration method according to the first aspect is implemented.

According to the image calibration method provided by the present disclosure, the calibration result of the fundus image is determined through the effective imaging region and the papilla region of the target fundus image, so that different fundus images obtained by different fundus imaging cameras may be calibrated and the calibration results obtained may be compared, thereby solving the problem of difficulty in comparing different fundus images captured by different fundus cameras, achieving measurement of related fundus characteristics, and facilitating related researches on comparison between different fundus images.

### BRIEF DESCRIPTION OF THE DRAWINGS

With reference to the accompanying drawings, a more detailed description of embodiments of the present disclosure will be given, so that the above and other purposes, features, and advantages of the present disclosure will become more evident. The accompanying drawings are used to provide a further understanding of the embodiments of the present disclosure and form a part of the specification, used in conjunction with the embodiments of the present disclosure to explain the disclosure, and do not constitute a limitation on the disclosure.

FIG. 1 is a schematic diagram of an application scenario of an embodiment of the present disclosure.

FIG. 2 is a flowchart of an image calibration method according to an embodiment of the present disclosure.

FIG. 3 is a flowchart of an image calibration method according to another embodiment of the present disclosure.

FIG. 4 is a flowchart of a step of determining, based on a target fundus image, a papilla region of the target fundus image according to an embodiment of the present disclosure.

FIG. 5 is a flowchart of a step of determining, based on a target fundus image, an effective imaging region of the target fundus image according to an embodiment of the present disclosure.

FIG. 6 is a flowchart of a step of determining, based on a target fundus image, an effective imaging region edge of the target fundus image according to an embodiment of the present disclosure.

FIG. 7 is a flowchart of an image processing method according to an embodiment of the present disclosure.

FIG. 8 is a schematic structural diagram of an image calibration apparatus according to an embodiment of the present disclosure.

FIG. 9 is a schematic structural diagram of an image processing apparatus according to an embodiment of the present disclosure.

FIG. 10 is a schematic structural diagram of an electronic device according to an embodiment of the present disclosure.

### DETAILED DESCRIPTION OF THE EMBODIMENTS

A clear and complete description of technical solutions in embodiments of the present disclosure will be given with reference to accompanying drawings. Obviously, the described embodiments are only a part of the embodiments of the present disclosure, and not all embodiments.

A fundus image may be used to understand morphology of a fundus structure and changes in the fundus structure. A precise measurement of the fundus structure is crucial for understanding morphological changes in fundus characteristics, disease diagnosis and treatment, so that the fundus image has become an essential auxiliary tool for clinical disease diagnosis and treatment. With the advent of the big data era, research on eye diseases and therapeutic effects by using a large volume of fundus images has been increasingly conducted.

However, in the production process of fundus cameras, different manufacturers set different imaging parameters for their cameras. As a result, sizes of the fundus characteristics captured by cameras with different imaging parameters may vary. Specifically, different fundus cameras have different imaging parameters, so that image resolution of different fundus cameras is different from each other, resulting in inconsistent sizes of fundus characteristics in fundus images of a same individual captured by different cameras. When an imaging width is also inconsistent, this inconsistency becomes even more pronounced, making it difficult to compare fundus characteristics captured by different cameras, which in turn poses challenges for related comparison researches, especially for multiple center clinical trial. Therefore, how to calibrate fundus images so that images captured by different cameras are comparable has become an urgent issue to be addressed.

To solve the above problems, an embodiment of the present disclosure provides an image calibration method to solve a problem of difficulty in comparing different fundus images captured by different fundus cameras.

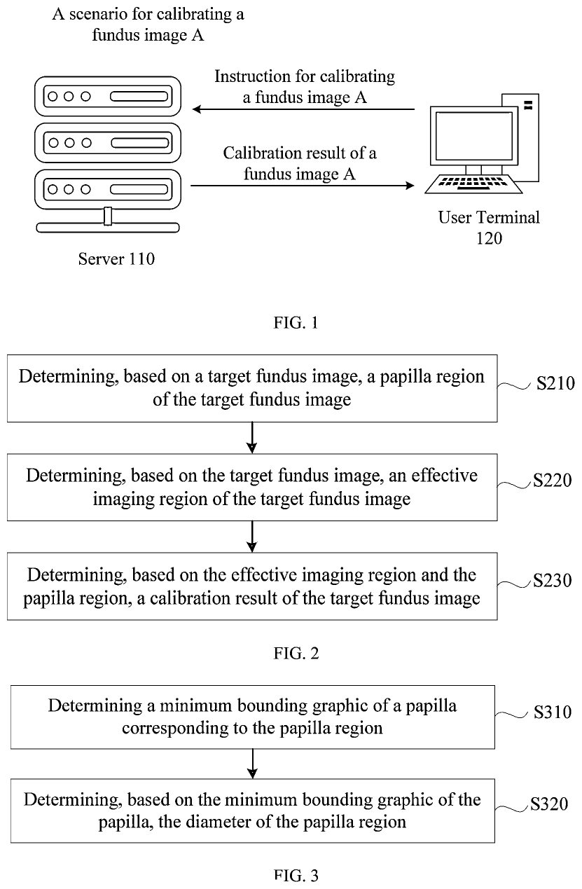

In the following a brief introduction to an application scenario of an embodiment of the present disclosure will be provided with reference to FIG. 1.

FIG. 1 is a schematic diagram of an application scenario of an embodiment of the present disclosure. As shown in FIG. 1, this scenario is a scene for calibrating a fundus image A (a target fundus image). Specifically, the scenario for calibrating the fundus image A (the target fundus image) includes a server 110 and a user terminal 120 in communication with the server 110. The server 110 is configured to implement an image calibration method mentioned in embodiments of the present disclosure.

Exemplarily, in practical applications, an user may send an instruction for calibrating the fundus image A to the server 110 through the user terminal 120. After receiving the instruction, the server 110 may process the fundus image A to obtain a papilla region and an effective imaging region of the fundus image A. Then, a calibration result of the fundus image A may be determined based on the papilla region and the effective imaging region of the fundus image A by the server 110. The calibration result of the fundus image A may be output to the user terminal 120 so that the user terminal 120 can present the calibration result of the fundus image A to the user.

Exemplarily, the user terminal 120 mentioned above includes but is not limited to computer terminals such as a desktop computer and a laptop, as well as mobile terminals such as a tablet and a mobile phone.

In the following, a brief introduction to an image calibration method of the present disclosure will be provided with reference to FIGS. 2 to 6.

FIG. 2 is a flowchart of an image calibration method according to an embodiment of the present disclosure. Exemplarily, the image calibration method provided by the embodiment of the present disclosure is executed by a server or a processor. As shown in FIG. 2, the image calibration method provided by the embodiment of the present disclosure may include the following steps.

Step S210: determining, based on a target fundus image, a papilla region of the target fundus image.

Exemplarily, the target fundus image refers to a fundus image to be calibrated, such as a fundus image of any one of a patient, a disease research volunteer, a normal person and the like.

Step S220: determining, based on the target fundus image, an effective imaging region of the target fundus image.

Exemplarily, the effective imaging region may be a region where a fundus structure is visualized, such as a region where the fundus structure is visualized in a color fundus image. The effective imaging region is generally located at a center of the target fundus image and is generally a circular region.

Step S230: determining, based on the effective imaging region and the papilla region, a calibration result of the target fundus image.

Exemplarily, the calibration result may be a calibration result of a fundus characteristic dimension of the target fundus image, and fundus characteristics may include an optic cup, a focus of infection, and so on. The calibration result may be a calibration result of dimensions of the optic cup, the focus of infection, and so on.

Exemplarily, the calibration result of the target fundus image may be determined based on a ratio of a diameter of the effective imaging region to a diameter of the papilla region, or based on a distance between a center position of a macula in the effective imaging region and a center position of the papilla region. The calibration result of the target fundus image includes a calibration result of a pixel unit dimension of the target fundus image and a fundus characteristic dimension of the target fundus image.

Since diameters of the effective imaging region and the papilla region are both within a preset range, the image calibration method mentioned in the embodiment of the present disclosure can calibrate fundus images of different cameras by determining the calibration result of the target fundus image based on the effective imaging region and the papilla region. The calibration results are obtained based on the effective imaging region and the papilla region, which makes the calibration results of the fundus images of different cameras comparable. Moreover, product parameters of existing fundus cameras only indicate parameters such as resolution and pixel count, but do not provide information of a dimension of each pixel. As the calibration result of the target fundus image in the embodiment of the present disclosure includes the calibration results of the pixel unit dimension of the target fundus image and the fundus characteristic dimension of the target fundus image, a problem of incapability in obtaining the dimension of each pixel for the fundus camera may be solved. In addition, the fundus characteristic dimensions such as vessel diameter and a dimension of a focus of infection may be obtained based on the pixel unit dimension, thereby facilitating quantitative evaluation of the fundus characteristics, enhancing accuracy, and providing scientific basis for subsequent diagnosis of fundus diseases.

In an embodiment of the present disclosure, the calibration result of the target fundus image is determined based on at least one of a ratio of a diameter of the effective imaging region to a diameter of the papilla region, a distance between a center position of a macula in the effective imaging region and a center position of the papilla region, and a ratio of an area of the effective imaging region to an area of the papilla region.

Exemplarily, the calibration result of the target fundus image may be determined based on the ratio of the diameter of the effective imaging region to the diameter of the papilla region, and a ratio of a diameter of a clinical effective imaging region to a diameter of an actual measured papilla region. Alternatively, the calibration result of the target fundus image may be determined based on a ratio of the distance between the center position of the macula in the effective imaging region and the center position of the papilla region to a distance between a true center position of a macula in the clinical effective imaging region and a center position of the actual measured papilla region. Alternatively, the calibration result of the target fundus image may be determined based on the ratio of the area of the effective imaging region to the area of the papilla region. Alternatively, the calibration result of the target fundus image may be determined based on an intersection of any two of the ratio of the diameter of the effective imaging region to the diameter of the papilla region, the distance between the center position of the macula in the effective imaging region and the center position of the papilla region, and the ratio of the area of the effective imaging region to the area of the papilla region. Alternatively, the calibration result of the target fundus image may be determined based on an intersection of the ratio of the diameter of the effective imaging region to the diameter of the papilla region, the distance between the center position of the macula in the effective imaging region and the center position of the papilla region, and the ratio of the area of the effective imaging region to the area of the papilla region.

Exemplarily, based on the calibration result determined based on the ratio of the diameter of the effective imaging region to the diameter of the papilla region, and the calibration result determined based on the distance between the center position of the macula in the effective imaging region and the center position of the papilla region, a final calibration result of the target fundus image may be obtained by intersecting the two calibration results. Alternatively, based on the calibration result determined based on the ratio of the diameter of the effective imaging region to the diameter of the papilla region, and the calibration result determined based on the distance between the center position of the macula in the effective imaging region and the center position of the papilla region, the final calibration result of the target fundus image may be determined by weighted summation to the calibration results determined.

Exemplarily, an imaging range of the effective imaging region obtained by a same fundus camera varies with different shooting angles. An imaging range of the clinical effective imaging region is consistent with the imaging range of the effective imaging region of the target fundus image. And the clinical effective imaging region and the target fundus image may be obtained under a same shooting angle. For example, the target fundus image is captured by a fundus camera at a shooting angle of 45°, and in this scenario, the clinical effective imaging region is also obtained at the same shooting angle of 45°.

Exemplarily, the shooting angle of the fundus imaging camera is less than or equal to 60°.

Exemplarily, the diameter of the clinical effective imaging region is obtained by calculating an average diameter of the effective imaging region of population. The diameter of the actual measured papilla region may be an average value of papilla diameters measured after relevant clinical dissection, or an average value of relevant data of the papilla diameter through manual absolute calibration, or an average value of a sum of the papilla diameters measured after relevant clinical dissection and the papilla diameters obtained through manual absolute calibration. Exemplarily, the population mentioned above refers to general population, which may include patients with non papilla lesions or healthy individuals. Meanwhile, although there are individual differences in papilla size, papilla sizes of population are similar and normally distributed. Therefore, the true papilla may can be determined by taking the average. Although the papilla size may be obtained through manual absolute calibration, extensive usage of manual absolute calibration in clinical research is limited due to its complexity and requirement for a certain level of expertise. Meanwhile, the manual absolute calibration cannot meet demand of current researches as the manual absolute calibration cannot be performed based on photos already taken.

Exemplarily, the distance between the center position of the macula and the center position of the papilla in the effective imaging region is determined by calculating an average distance between the center position of the macula in the effective imaging region and the center position of the papilla of population; or an average value of relevant data of distance obtained through manual absolute calibration.

According to the image calibration method mentioned in the embodiment of the present disclosure, the fundus image is calibrated based on the diameter of the effective imaging region and the diameter of the papilla region, and/or based on the distance between the center position of the macula in the effective imaging region and the center position of the papilla region to obtain unified calibration parameters with reference to real measurement values, so that calibration results of fundus characteristic of different fundus images captured by different cameras may be comparable, thereby facilitating quantitative evaluation of fundus characteristics, improving accuracy, and providing scientific basis for subsequent diagnosis of fundus diseases.

FIG. 3 is a flowchart of an image calibration method according to another embodiment of the present disclosure. As shown in FIG. 3, before a step of determining, based on at least one of a ratio of a diameter of the effective imaging region to a diameter of the papilla region, a distance between a center position of a macula in the effective imaging region and a center position of the papilla region, and a ratio of an area of the effective imaging region to an area of the papilla region, a calibration result of a target fundus image, the image calibration method further includes the following steps.

Step S310: determining a minimum bounding graphic of a papilla corresponding to the papilla region.

Exemplarily, the minimum bounding graphic of the papilla may be a minimum bounding circle of the papilla, a minimum bounding ellipse of the papilla, or a minimum bounding rectangle of the papilla.

Step S320: determining, based on the minimum bounding graphic of the papilla, the diameter of the papilla region.

Exemplarily, the diameter of the papilla region is determined based on a diameter of the minimum bounding circle of the papilla. Alternatively, the diameter of the papilla region is determined based on a major axis of the minimum bounding ellipse of the papilla. Alternatively, the diameter of the papilla region is determined based on a major axis of the minimum bounding rectangle of the papilla.

According to the embodiment of the present disclosure, the diameter of the papilla region is determined through the minimum bounding graphic of the papilla, so that the diameter of the papilla region is more accurate, thereby improving accuracy of the image calibration result.

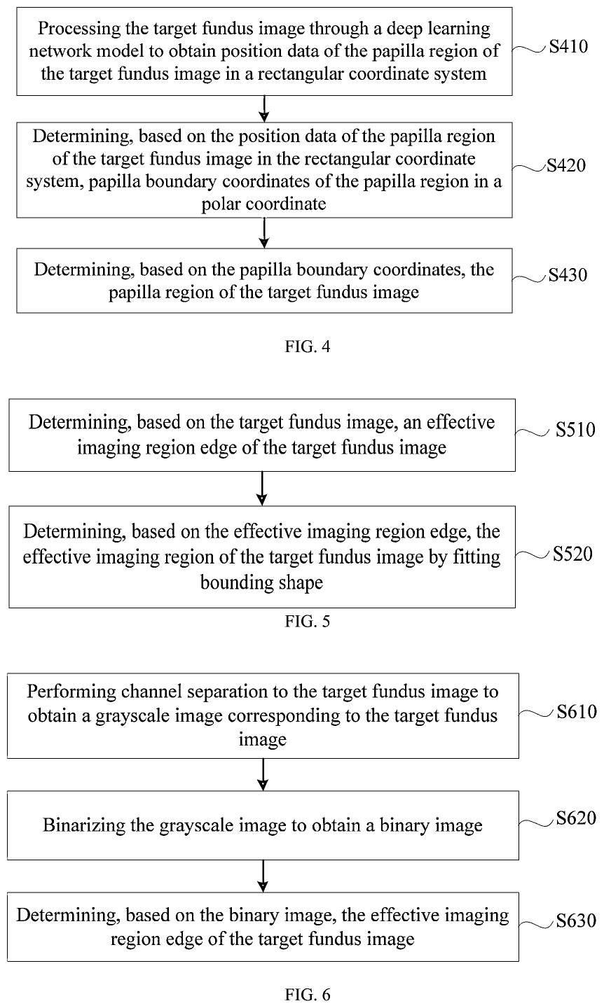

FIG. 4 is a flowchart of a step of determining, based on a target fundus image, a papilla region of the target fundus image according to an embodiment of the present disclosure. As shown in FIG. 4, the step of determining, based on the target fundus image, the papilla region of the target fundus image provided in the embodiment of the present disclosure may include the following steps.

Step S410: processing the target fundus image through a deep learning network model to obtain position data of the papilla region of the target fundus image in a rectangular coordinate system.

Exemplarily, a position image of the papilla region is obtained by processing the target fundus image through the deep learning network model, and the position data of the papilla region of the target fundus image in the rectangular coordinate system is obtained based on the position image of the papilla region.

Step S420: determining, based on the position data of the papilla region of the target fundus image in the rectangular coordinate system, papilla boundary coordinates of the papilla region in a polar coordinate.

Exemplarily, two-dimensional polar coordinate transformation may be performed based on a radius of the papilla region of the target image in the rectangular coordinate system. By performing the polar coordinate transformation to the position data in the rectangular coordinate system, the papilla boundary of the papilla region may be transformed into a clear and visible curve in a horizontal direction.

Step S430: determining, based on the papilla boundary coordinates, the papilla region of the target fundus image.

Exemplarily, the papilla region of the target fundus image is determined based on the the curve in the horizontal direction obtained above.

According to the embodiment of the present disclosure, a clear and visible curve in a horizontal direction may be obtained through the polar coordinate transformation, so that accuracy of papilla edge segmentation may be improved, thereby enhancing precision of the papilla region in the target fundus image and improving accuracy of the image calibration result.

In an embodiment of the present disclosure, the method of determining, based on the target fundus image, the papilla region of the target fundus image may also include: processing the target fundus image through computer vision technology to obtain the papilla region of the target fundus image. Exemplarily, by using the computer vision technology, fundus image content may be detected based on computer vision attention mechanism to identify a position of a papilla and determine the papilla region of the target fundus image.

In an embodiment of the present disclosure, the method of determining, based on the target fundus image, the papilla region of the target fundus image may also include: processing the target fundus image through deep learning segmentation network to obtain the papilla region of the target fundus image. Exemplarily, by using a trained deep learning segmentation network, the target fundus image may be processed to segment the papilla region of the target fundus image to obtain the papilla region of the target fundus image.

According to the present disclosure, a method for processing the target fundus image through the computer vision technology is provided, so that the papilla region of the target fundus image may be obtained directly, thereby simplifying acquisition of the diameter of the papilla region, and reducing complexity of the image calibration process.

FIG. 5 is a flowchart of a step of determining, based on a target fundus image, an effective imaging region of the target fundus image according to an embodiment of the present disclosure. As shown in FIG. 5, the determining, based on the target fundus image, the effective imaging region of the target fundus image provided in the embodiment of the present disclosure may include the following steps.

Step S510: determining, based on a target fundus image, an effective imaging region edge of the target fundus image.

Exemplarily, the effective imaging region edge of the target fundus image may be determined, based on the target fundus image, by using a gradient threshold or an edge detection operator (such as Canny edge detection operator). Exemplarily, an edge of the papilla is a region with the fastest change in a longitudinal direction. Therefore, by using the gradient threshold, some noise and edge overlap may be effectively eliminated, and then the edge of the papilla may be obtained by using maximum gradient edge linking.

Step S520: determining, based on the effective imaging region edge, the effective imaging region of the target fundus image by fitting bounding shape.

Exemplarily, the effective imaging region may be directly obtained through the target fundus image, and then the effective imaging region may be processed to obtain the effective imaging region edge. The effective imaging region edge may be processes by fitting bounding shape to obtain a processed effective imaging region of the fundus image, and the effective imaging region of the fundus image may be determined based on the processed effective imaging region of the fundus image.

Exemplarily, the determining the effective imaging region of the target fundus image by fitting bounding shape may include: performing Hough transform to the effective imaging region of the target fundus image. Specifically, circular Hough transform is preformed to the effective imaging region edge of the target fundus image, and then a circle with the most votes is determined as the effective imaging region of the target fundus image. A process of Hough transform is similar to a process of election voting. A candidate circle is determined through three points and intersection points of all points on the image edge and the candidate circles are determined as votes. A candidate circle with the most votes may be determined as the effective imaging region of the target fundus image.

According to the embodiment of the present disclosure, the effective imaging region edge of the target fundus image is obtained by using the gradient threshold or the Canny edge detection operator, so that the effective imaging region edge may be more accurate, thereby improving accuracy of calibration. In addition, through the Hough transform, a radius of the effective imaging region may be accurately identified when the effective imaging region is not a complete circle, so that requirements of image calibration for the image may be reduced, and different fundus images of different cameras may be processed, thereby increasing applicability of image calibration.

FIG. 6 is a flowchart of a step of determining, based on a target fundus image, an effective imaging region edge of the target fundus image according to an embodiment of the present disclosure. As shown in FIG. 6, the determining, based on the target fundus image, the effective imaging region edge of the target fundus image provided in the embodiment of the present disclosure may include the following steps.

Step S610: performing channel separation to the target fundus image to obtain a grayscale image corresponding to the target fundus image.

Exemplarily, one or a combination of three color channels including Red, Green, and Blue may be selected as an extraction channel. Then channel separation is performed to the target fundus image. In addition, one or a combination of attributes including Hue, Saturation, and Lightness may also be selected as an extraction channel to perform channel separation to the target fundus image. Alternatively, a combination of two or more of channels including Red, Green, Blue, Hue, Saturation, and Lightness may be selected as the extraction channel. For example, a combination of Red and Hue may be selected as the extraction channel to perform channel separation to the target fundus image.

Step S620: binarizing the grayscale image to obtain a binary image.

Exemplarily, taking ⅓ of a channel grayscale mean value as a threshold, the grayscale image is binarized to obtain the binary image.

Step S630: determining, based on the binary image, the effective imaging region edge of the target fundus image.

According to the embodiment of the present disclosure, the effective imaging region edge of the target fundus image is determined through channel separation and image binarization. Thus, the edge of the effective imaging region of the target fundus image may be more accurate, thereby improving accuracy of the image calibration result.

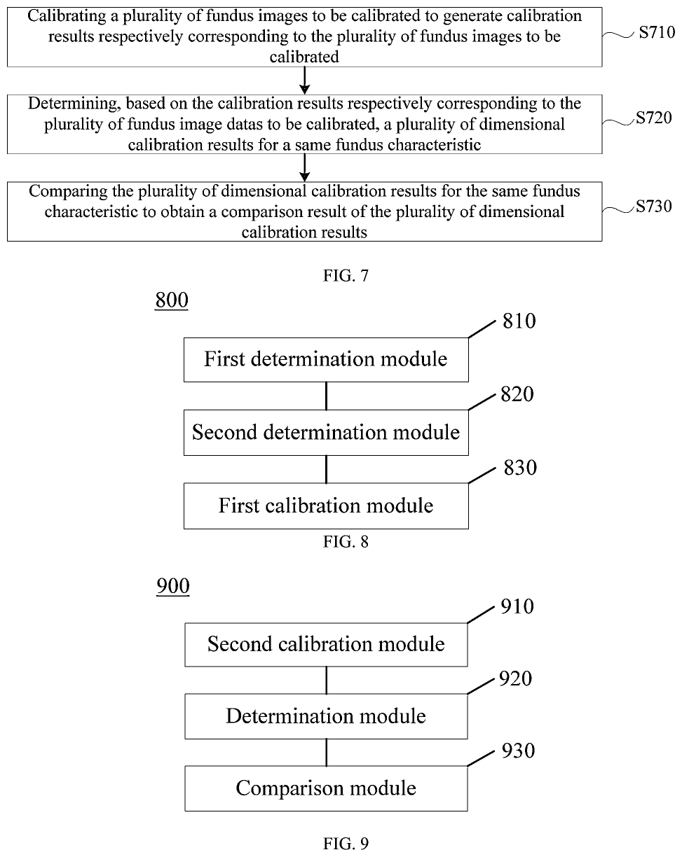

FIG. 7 is a flowchart of an image processing method according to an embodiment of the present disclosure. As shown in FIG. 7, the image processing method provided in the embodiment of the present disclosure may include the following steps.

Step S710: calibrating a plurality of fundus images to be calibrated to generate calibration results respectively corresponding to the plurality of fundus images to be calibrated.

Exemplarily, the plurality of fundus images to be calibrated may be calibrated by the image calibration method mentioned in any one of the above embodiments to generate calibration results respectively corresponding to the plurality of fundus images to be calibrated. Exemplarily, the plurality of fundus images to be calibrated may be fundus images captured by different cameras and at different times for the same person, or fundus images captured by different cameras and at different times for different persons.

Step S720: determining, based on the calibration results respectively corresponding to the plurality of fundus images to be calibrated, a plurality of dimensional calibration results for a same fundus characteristic.

Exemplarily, the plurality of dimensional calibration results for a same fundus characteristic in the plurality of fundus images may be determined based on the calibration results respectively corresponding to the plurality of fundus images captured by different cameras and at different times for the same person. For example, different dimensional calibration results for a focus of infection in different fundus images may be determined based on the calibration results.

Step S730: comparing the plurality of dimensional calibration results for the same fundus characteristic to obtain a comparison result of the plurality of dimensional calibration results.

Exemplarily, if the dimensional calibration results of a focus of infection in different fundus images are different, the fundus characteristics are compared based on the plurality of dimensional calibration results for the same fundus characteristic of the plurality of fundus images in a plurality of fundus image data obtained above. By comparing dimensions of the focus of infection in the plurality of fundus images of a same patient, development of the patient's focus of infection may be determined, thereby enabling comparison of different fundus image data captured by different cameras and helping to complete related researches.

According to the embodiment of the present disclosure, a plurality of fundus images to be calibrated may be calibrated by the image calibration method mentioned in any one of the above embodiments. Since the calibration results of the plurality of fundus images are obtained based on real clinical data and have a unified calibration standard, the calibration results of the plurality of fundus images may be comparable. In addition, in the embodiment of the present disclosure, the calibration results respectively corresponding to the plurality of fundus images are used to determine the plurality of dimensional calibration results for the same fundus characteristic (such as sizes of vessel diameter, focus of infection, optic cup, and so on), and the plurality of dimensional calibration results for the same fundus characteristic are compared to determine morphological changes of the fundus characteristic, thereby providing assistance for a related research of comparing different fundus images.

FIG. 8 is a schematic structural diagram of an image calibration apparatus according to an embodiment of the present disclosure. As shown in FIG. 8, the image calibration apparatus 800 provided in the embodiment of the present disclosure includes: a first determination module 810, a second determination module 820, and a first calibration module 830. The first determination module 810 is configured to determine, based on a target fundus image, a papilla region of the target fundus image; the second determination module 820 is configured to determine, based on the target fundus image, an effective imaging region of the target fundus image, where the effective imaging region includes a region where a fundus structure is visualized; and the calibration module first 830 is configured to determine, based on the effective imaging region and the papilla region, a calibration result of the target fundus image, where the calibration result includes a calibration result of a pixel unit dimension of the target fundus image and a calibration result of a fundus characteristic dimension of the target fundus image.

In some embodiments, the first determination module 810 is further configured to determine a minimum bounding graphic of a papilla corresponding to the papilla region; and determine, based on the minimum bounding graphic of the papilla, a diameter of the papilla region.

In some embodiments, the first determination module 810 is further configured to process the target fundus image through a deep learning network model to obtain position data of the papilla region of the target fundus image in a rectangular coordinate system; perform polar coordinate transformation to the position data in the rectangular coordinate system and determine a papilla boundary coordinates of the papilla region in a polar coordinate; and determine, based on the papilla boundary coordinates, the papilla region of the target fundus image.

In some embodiments, the first determination module 810 is further configured to process the target fundus image through computer vision technology to obtain the papilla region of the target fundus image.

In some embodiments, the first determination module 810 is further configured to process the target fundus image through deep learning segmentation network to obtain the papilla region of the target fundus image.

In some embodiments, the second determination module 820 is further configured to determine, based on the target fundus image, an effective imaging region edge of the target fundus image; and determine, based on the effective imaging region edge, the effective imaging region of the target fundus image by fitting bounding shape.

In some embodiments, the second determination module 820 is further configured to perform channel separation to the target fundus image to obtain a grayscale image corresponding to the target fundus image; binarize the grayscale image to obtain a binary image; and determine, based on the binary image, the effective imaging region edge of the target fundus image.

In some embodiments, the first calibration module 830 is further configured to determine, based on a ratio of a diameter of the effective imaging region to a diameter of the papilla region, the calibration result of the target fundus image.

In some embodiments, the first calibration module 830 is further configured to determine, based on a distance between a center position of a macula in the effective imaging region and a center position of the papilla region, the calibration result of the target fundus image.

In some embodiments, the first calibration module 830 is further configured to determine, based on a ratio of an area of the effective imaging region to an area of the papilla region, the calibration result of the target fundus image.

In some embodiments, the first calibration module 830 is further configured to determine, based on the ratio of a diameter of the effective imaging region to a diameter of the papilla region, and the distance between a center position of a macula in the effective imaging region and a center position of the papilla region, the calibration result of the target fundus image.

Exemplarily, based on the calibration result determined based on the ratio of the diameter of the effective imaging region to the diameter of the papilla region, and the calibration result determined based on the distance between the center position of the macula in the effective imaging region and the center position of the papilla region, a final calibration result of the target fundus image is obtained by intersecting the two calibration results. Alternatively, based on the calibration result determined based on the ratio of the diameter of the effective imaging region to the diameter of the papilla region, and the calibration result determined based on the distance between the center position of the macula in the effective imaging region and the center position of the papilla region, the final calibration result of the target fundus image is determined by weighted summation to the calibration results determined.

In some embodiments, the first calibration module 830 is further configured to determine, based on an intersection of any two of the ratio of the diameter of the effective imaging region to the diameter of the papilla region, the distance between the center position of the macula in the effective imaging region and the center position of the papilla region, and the ratio of the area of the effective imaging region to the area of the papilla region, the calibration result of the target fundus image.

In some embodiments, the first calibration module 830 is further configured to determine, based on an intersection of the ratio of the diameter of the effective imaging region to the diameter of the papilla region, the distance between the center position of the macula in the effective imaging region and the center position of the papilla region, and the ratio of the area of the effective imaging region to the area of the papilla region, the calibration result of the target fundus image.

FIG. 9 is a schematic structural diagram of an image processing apparatus according to an embodiment of the present disclosure. As shown in FIG. 9, the image processing apparatus 900 provided in the embodiment of the present disclosure includes: a second calibration module 910, a determination module 920, and a comparison module 930. The second calibration module 910 is configured to calibrate a plurality of fundus images to be calibrated by the image calibration method mentioned in any one of embodiments described above to generate calibration results respectively corresponding to the plurality of fundus images to be calibrated; the determination module 920 is configured to determine, based on the calibration results respectively corresponding to the plurality of fundus images to be calibrated, a plurality of dimensional calibration results for a same fundus characteristic; and the comparison module 930 is configured to compare the plurality of dimensional calibration results for the same fundus characteristic to obtain a comparison result of the plurality of dimensional calibration results.

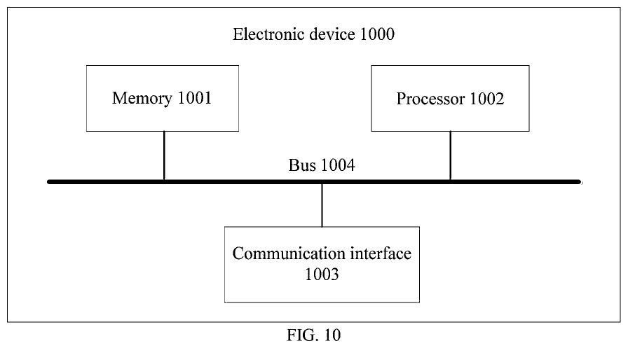

FIG. 10 is a schematic structural diagram of an electronic device according to an embodiment of the present disclosure. The electronic device 1000 shown in FIG. 10 (which can specifically be a computer device) includes a memory 1001, a processor 1002, a communication interface 1003, and a bus 1004, where the memory 1001, the processor 1002, and the communication interface 1003 are connected to each other through the bus 1004 for communication.

The memory 1001 may be Read Only Memory (ROM), static storage device, dynamic storage device, or Random Access Memory (RAM). The memory 1001 can store programs, and when the programs stored in memory 1001 are executed by the processor 1002, the processor 1002 and the communication interface 1003 are used for executing steps in the image calibration method provided by the embodiments of the present disclosure.

The processor 1002 may be a general-purpose Central Processing Unit (CPU), microprocessor, Application Specific Integrated Circuit (ASIC), Graphics Processing Unit (GPU), or one or more integrated circuits to execute relevant programs, thereby implementing the functions required for each unit in the image calibration apparatus of the embodiments of the present disclosure.

The processor 1002 may also be an integrated circuit chip with signal processing capabilities. In the implementation process, the various steps of the image calibration method in the present disclosure can be accomplished through integrated logic circuits in hardware within the processor 1002 or software instructions. The aforementioned processor 1002 may also be a general-purpose processor, Digital Signal Processor (DSP), Application-Specific Integrated Circuit (ASIC), Field-Programmable Gate Array (FPGA) or other programmable logic devices, discrete gates or transistor logic devices, or discrete hardware components, and may implement or execute methods, steps, and logic block diagrams provided in the embodiments of the present disclosure. The general-purpose processor may be a microprocessor, or any conventional processor, etc. The steps of the method in the embodiments of the present disclosure may be directly implemented by a hardware decoding processor, or they can be executed by a combination of hardware and software modules within the decoding processor. The software modules can be stored in mature storage medium within this field, such as random access memory (RAM), flash memory, read-only memory (ROM), programmable read-only memory (PROM), electrically erasable programmable read-only memory (EEPROM), registers, and so on. The storage medium is located in the memory 1001, where the processor 1002 reads the information from the memory 1001 and, in conjunction with its hardware, performs the functions required by the units included in the image calibration apparatus of the embodiments of the present disclosure, or executes the image calibration method provided by the embodiments of the present disclosure.

The communication interface 1003 uses a transceiver or similar transmission and reception devices to facilitate communication between the electronic device 1000 and other devices or communication networks. For example, image data to be calibrated can be obtained through the communication interface 1003.

The bus 1004 may include pathways for transmitting information between various components of the electronic device 1000 (such as, the memory 1001, the processor 1002, and the communication interface 1003).

It should be noted that although FIG. 10 shows an electronic device 1000 with only a memory, a processor, and a communication interface, in the actual implementation process, those skilled in the art should understand that the electronic device 1000 also includes other components necessary for normal operation. At the same time, depending on specific needs, those skilled in the art should understand that the electronic device 1000 may also include hardware components for achieving additional functions. Furthermore, those skilled in the art should understand that the electronic device 1000 may only include the components necessary to implement the embodiments of the present disclosure, and does not necessarily include all the components shown in FIG. 10.

Those skilled in the art can realize that, by combining the unit and algorithm steps described in the examples of the embodiments of the present disclosure, they can be implemented in electronic hardware, or a combination of computer software and electronic hardware. Whether these functions are executed in hardware or software depends on the specific application and design constraints of the technical solution. Professional technicians can use different methods for each specific application to implement the described functions, but such implementations should not be considered beyond the scope of the present disclosure.

Those skilled in the art can clearly understand that, for the convenience and conciseness of description, the specific working processes of the systems, apparatuses, and units described above can refer to the corresponding processes in the aforementioned method embodiments, which will not be repeated herein.

In the several embodiments of the present disclosure, it should be understood that the systems, apparatuses, and methods disclosed can be realized in other ways. For example, the apparatus embodiments described above are merely illustrative; for instance, the division of units is merely a logical functional division. In actual implementation, there can be other ways of division, such as some units or components being combined or integrated into another system, or some features being omitted or not executed. Additionally, the coupling or direct coupling or communication connections shown or discussed between each other can be indirect couplings or communication connections through some interfaces, apparatuses, or units, and can be in the form of electrical, mechanical, or other forms.

The units described as separate components may or may not be physically separated. The components displayed as units may or may not be physical units, meaning they can be located in one place or distributed across multiple network units. Depending on actual needs, some or all of these units can be selected to achieve the objectives of the present embodiments' scheme.

Additionally, each functional unit in each embodiment of the present disclosure may be integrated into a single processing unit, or each unit may physically exist separately, or two or more units may be integrated in one unit.

In addition, the embodiments of the present disclosure may also be a computer-readable storage medium, on which computer executable instructions are stored, where when the executable instructions are executed by a processor, the steps in the methods described above in this specification according to method described in various embodiments of the present disclosure is implemented. The function may be stored in a computer-readable storage medium if it is realized in the form of software functional unit, and sold or used as an independent product. Based on this, the technical solutions in essence, or the contributions to the conventional technology, or a part of the technical solutions, of the present application may be embodied in the form of software products, which are stored in the storage medium, and include several instructions to enable a computer device (which may be a personal computer, a server, a network device, etc.) to perform all or part of the steps of the method described in each embodiment of the present application. The readable storage medium may include, but is not limited to, systems, apparatuses, or devices of electricity, magnetism, light, electromagnetism, infrared, or semiconductors, or any combination thereof. More specific examples of the aforementioned storage media (a non-exhaustive list) include: USB flash drives, portable hard drives, Read Only Memory (ROM), Random Access Memory (RAM), magnetic disks, or optical discs, and any other medium capable of storing program code, or any suitable combination thereof.

The above description is merely specific embodiments of the present disclosure, but the protection scope of the present disclosure is not limited to this. Any skilled in the art familiar with the technical field can easily think of variations or substitutions within the technical scope disclosed by the present disclosure, and all should be covered within the protection scope of the present disclosure. Therefore, the protection scope of the present disclosure should be determined by the scope of the claims.

What is claimed is:

1. An image calibration method, comprising:
determining, based on a target fundus image, a papilla region of the target fundus image;
determining, based on the target fundus image, an effective imaging region of the target fundus image, wherein the effective imaging region comprises a region where a fundus structure is visualized; and
determining, based on the effective imaging region and the papilla region, a calibration result of the target fundus image, wherein the calibration result comprises a calibration result of a pixel unit dimension of the target fundus image and a calibration result of a fundus characteristic dimension of the target fundus image.

2. The image calibration method according to claim 1, wherein the determining, based on the effective imaging region and the papilla region, the calibration result of the target fundus image comprises:
determining, based on at least one of a ratio of a diameter of the effective imaging region to a diameter of the papilla region, a distance between a center position of a macula in the effective imaging region and a center position of the papilla region, and a ratio of an area of the effective imaging region to an area of the papilla region, the calibration result of the target fundus image.

3. The image calibration method according to claim 2, wherein before the determining, based on at least one of a ratio of a diameter of the effective imaging region to a diameter of the papilla region, a distance between a center position of a macula in the effective imaging region and a center position of the papilla region, and a ratio of an area of the effective imaging region to an area of the papilla region, the calibration result of the target fundus image, the image calibration method further comprises:
determining a minimum bounding graphic of a papilla corresponding to the papilla region; and
determining, based on the minimum bounding graphic of the papilla, the diameter of the papilla region.

4. The image calibration method according to claim 3, wherein the minimum bounding graphic of the papilla comprises at least one of a minimum bounding circle of the papilla, a minimum bounding ellipse of the papilla, and a minimum bounding rectangle of the papilla.

5. The image calibration method according to claim 4, wherein
when the minimum bounding graphic of papilla comprises the minimum bounding circle of the papilla, the determining, based on the minimum bounding graphic of the papilla, the diameter of the papilla region comprises: determining, based on a diameter of the minimum bounding circle of the papilla, the diameter of the papilla region;
when the minimum bounding graphic of papilla comprises the minimum bounding ellipse of the papilla, the determining, based on the minimum bounding graphic of the papilla, the diameter of the papilla region comprises: determining, based on a long axis of the minimum bounding ellipse of the papilla, the diameter of the papilla region; and
when the minimum bounding graphic of papilla comprises the minimum bounding rectangle of the papilla, the determining, based on the minimum bounding graphic of the papilla, the diameter of the papilla region comprises: determining, based on a long axis of the minimum bounding rectangle of the papilla, the diameter of the papilla region.

6. The image calibration method according to claim 1, wherein the determining, based on the target fundus image, the papilla region of the target fundus image comprises:
processing the target fundus image through a deep learning network model to obtain position data of the papilla region of the target fundus image in a rectangular coordinate system;
performing polar coordinate transformation to the position data in the rectangular coordinate system, and determining papilla boundary coordinates of the papilla region in a polar coordinate; and
determining, based on the papilla boundary coordinates, the papilla region of the target fundus image.

7. The image calibration method according to claim 1, wherein the determining, based on the target fundus image, the papilla region of the target fundus image comprises:
processing the target fundus image through computer vision technology to obtain the papilla region of the target fundus image.

8. The image calibration method according to claim 1, wherein the determining, based on the target fundus image, the papilla region of the target fundus image comprises:
processing the target fundus image through deep learning segmentation network to obtain the papilla region of the target fundus image.

9. The image calibration method according to claim 1, wherein the determining, based on the target fundus image, the effective imaging region of the target fundus image comprises:
determining, based on the target fundus image, an effective imaging region edge of the target fundus image; and
determining, based on the effective imaging region edge, the effective imaging region of the target fundus image by fitting bounding shape.

10. The image calibration method according to claim 9, wherein the determining, based on the effective imaging region edge, the effective imaging region of the target fundus image by fitting bounding shape comprises:
performing circular Hough transform to the effective imaging region edge of the target fundus image; and
determining a circle with the most votes as the effective imaging region of the target fundus image.

11. The image calibration method according to claim 9, wherein the determining, based on the target fundus image, the effective imaging region edge of the target fundus image comprises:
determining, based on the target fundus image, an effective imaging region edge of the target fundus image through a gradient threshold or an edge detection operator.

12. The image calibration method according to claim 9, wherein the determining, based on the target fundus image, the effective imaging region edge of the target fundus image comprises:
performing channel separation to the target fundus image to obtain a grayscale image corresponding to the target fundus image;
binarizing the grayscale image to obtain a binary image; and
determining, based on the binary image, the effective imaging region edge of the target fundus image.

13. The image calibration method according to claim 12, wherein the performing channel separation to the target fundus image to obtain the grayscale image corresponding to the target fundus image comprises:
selecting one or a combination of red, green, and blue color channels as an extraction channel, and performing channel separation to the target fundus image to obtain the grayscale image corresponding to the target fundus image.

14. The image calibration method according to claim 12, wherein the performing channel separation to the target fundus image to obtain the grayscale image corresponding to the target fundus image comprises:
selecting one or a combination of hue, saturation, and brightness attributes as an extraction channel, and performing channel separation to the target fundus image to obtain the grayscale image corresponding to the target fundus image.

15. The image calibration method according to claim 12, wherein the performing channel separation to the target fundus image to obtain the grayscale image corresponding to the target fundus image comprises:
selecting a combination of two or more of red, green, and blue color channels, and hue, saturation, and brightness attributes as an extraction channel, and performing channel separation to the target fundus image to obtain the grayscale image corresponding to the target fundus image.

16. An image processing method, comprising:
calibrating a plurality of fundus images to be calibrated by the image calibration method according to claim 1 to generate calibration results respectively corresponding to the plurality of fundus images to be calibrated;
determining, based on the calibration results respectively corresponding to the plurality of fundus images to be calibrated, a plurality of dimensional calibration results for a same fundus characteristic; and
comparing the plurality of dimensional calibration results for the same fundus characteristic to obtain a comparison result of the plurality of dimensional calibration results.

17. An electronic device, comprising:
a processor; and
a memory, configured to store executable instructions of the processor, wherein
the processor is configured to implement the image calibration method according to claim 1.

18. A non-transitory computer-readable storage medium, on which computer executable instructions are stored, wherein when the executable instructions are executed by a processor, the image calibration method according to claim 1 is implemented.
